(19) United States
(12) Patent Application Publication
(10) Pub. No.: US 2010/0049207 A1
(43) Pub. Date: Feb. 25, 2010

(54) Jones tube inserter

(76) Inventor: Nicolas A. Turmes, JR., Plantation, FL (US)

Correspondence Address: W. THOMAS TIMMONS, 1320 PRUDENTIAL DRIVE, SUITE 208, DALLAS, TX, 75235-4117, US

(21) Appl. No.: 12/229,423

(22) Filed: Aug. 22, 2008

(51) Int. Cl. A61B 17/00

(52) U.S. Cl. 606/108

(57) ABSTRACT

A Jones tube inserter is disclosed which includes a guide line and a bullet affixed to one end of the guide line. The bullet accommodates a Jones tube behind it on the guide line. The bullet is slanted to accommodate the beveled end of the Jones tube. The base of the bullet forms a square which is approximately the size of the inside diameter of the Jones tube, whereby the square formed by the bullet fits snugly into the Jones tube. A procedure for inserting a Jones tube, includes the steps of a. anesthetize the tissue and surrounding tissue at the site of the conjunctivodacryocystorhinostomy; b. remove the old Jones Tube, if any; c. insert a Jones Gold Dilator into the opening of the tissue where the conjunctivodacryocystorhinostomy has been performed and through the hole in the nasal bone to maintain the integrity; d. add an antihistamine, if necessary, to shrink the surrounding tissue and allowed to set; e. reinsert the Jones Gold Dilator into the surgically formed opening; f. put the Jones tube on the inserter; g. push the Jones tube to the base of the line, and then turn it to align the beveling in the base of the bullet of the inserter with the Jones tube beveling; h. push the Jones tube towards the bullet so it will be securely held in place by a sleeve during the actual insertion; and i. remove the Jones Gold Dilator. Further steps can be included.

### CROSS-REFERENCE TO RELATED APPLICATIONS

### Statement Regarding Federally Sponsored Research or Development

Not Applicable

### INCORPORATION-BY-REFERENCE OF MATERIAL SUBMITTED ON A COMPACT DISC

Not Applicable

### BACKGROUND OF THE INVENTION

1. Field of the Invention

The present invention relates generally to the field of the treatment of tear duct problems and, in one of its aspects, to an improved method and device to aid in Conjunctivodacryocystorhinostomy surgery.

2. Description of Related Art

Conjunctivodacryocystorhinostomy surgery provides relief to millions of people every year. People suffer from tear duct problems or from the complete removal of the tear duct due to trauma or physiological problems in the surrounding tissue involving the canalicular system.

Lester T. Jones and Gunter Weiss, in 1961, developed a solution for tear duct problems. They developed and designed a specialized glass tube for insertion into corner of the eye, with a funnel like end, next to the eye to drain the eyes of excess liquid or tears. This newly developed tube was called the “Lester T. Jones tear duct tube” (“Jones Tube”).

The Jones Tube comes in a variety of lengths, diameters and angles in order to accommodate the patients needs. The Jones Tube replaces the destroyed or removed lacrimal drainage ducts, the canaliculi system and/or the tear sack.

The Jones Tube procedure includes the following steps:
1. The tissue, eye and surrounding tissue at the site of the conjunctivodacryocystorhinostomy is given anesthetic drops.
2. The old Jones Tube is then removed using forceps. This is relatively easy and causes very little pain or discomfort to the patient.
3. A Jones Gold Dilator, covered with an anesthetic material, is then inserted into the opening of the tissue where the conjunctivodacryocystorhinostomy has been performed and through the hole in the nasal bone to maintain the integrity and to help anesthetize the muscosa of the tissue while the Jones Tube is being prepared for insertion.
4. The Jones Gold Dilator is then removed after approximately five minutes.
5. An antihistamine is placed by means of a Q-tip into the corner of the patient's eye where the new tube is to be inserted in order to shrink the surrounding tissue. It is allowed to set for approximately five (5) minutes.
6. The Jones Gold Dilator is then reinserted into the surgically formed opening, while the Jones Tube is being prepared for insertion.
7. The Jones Gold Dilator is then removed and the guide wire with the Jones Tube attached is inserted into the hole.
8. The Jones Tube is then pushed down the wire and pushed into the hole. It is pressed until it sets itself in the bone.
9. Once the Jones Tube is set, the procedure is completed and the guide wire is then removed.

The procedure should be done every four (4) to six (6) months to keep material from encrusting on the Jones Tube, minimize bacterial growth or buildup on the Jones Tube and so hole in the nasal bone does not get smaller. It is, at times, a very painful procedure. Therefore, the patient tends not to have it done on a regularly scheduled basis. The hole in the nasocavity tends to grow smaller, the tissue tends to overgrow around the Jones Tube and the eye can become red and inflamed due to the excess growth of bacteria.

Even if the surrounding tissue is given anesthetic and/or shrunk by the use of antihistamine, it is still very painful and uncomfortable to reinsert the new Jones Tube into the surgically formed opening.

The Jones Gold Dilator goes into the hole left by the removed Jones Tube to keep the tissue and the hole ready to accept the new Jones Tube. Once the Jones Gold Dilator is removed and the wire with the new Jones Tube is put into place, the tissue tends to close around the wire making it difficult to set the new Jones Tube.

The physician has to force the Jones Tube past the tissue into the hole. The leading end of the Jones Tube is beveled but it is difficult to pass through the mucosal lined osteotomy.

There needs to be a way to help alleviate the trauma and pain of this procedure.

U.S. Pat. No. 3,042,044 (D. S. Sheridan) shows a tube that, like the Jones Tube, is designed for temporary lodgment within the human body.

U.S. Pat. No. 3,726,284 (Ralph W. Parker) shows a drainage tube to be permanently embedded to replace the destroyed or removed lacrimal drainage ducts.

### BRIEF SUMMARY OF THE INVENTION

The present invention is intended to help alleviate the trauma and the pain of the prior art procedure.

The Jones Tube inserter apparatus of the present invention is approximately 115 mm in length. It is made of metal, precious metal, plastic, rubber, glass, Teflon or a polymer material, or some combination of such materials.

The distal end or tip is shaped similarly to the Jones Gold Dilator and is from 0.1 mm to 5 mm in length, the bullet has similar diameter to the Jones Tube. The end or base of the bullet is somewhat slanted to accommodate the beveled end of the Jones Tube.

At the base of the bullet, opposite the insertion end, is a line (metal, precious metal, plastic, rubber, glass, Teflon or a polymer material or some combination of such materials) that can be a solid apparatus or screwed in depending upon the manufacturing process used. Also at the base of the bullet is a small square which is approximately the size of the inside diameter of the Jones Tube. This is to put into the Jones Tube. This keeps the Jones Tube from sliding from one side to the other during insertion.

These and other objects, advantages and features of this invention will be apparent from the following description taken with reference to the accompanying drawing, wherein is shown a preferred embodiment of the invention.

### BRIEF DESCRIPTION OF THE SEVERAL VIEWS OF THE DRAWING

FIG. 1 is a side view of a Jones tube inserter according to the present invention;

FIG. 2 is a side view of the Jones tube inserter of FIG. 1 with a Jones tube in place; and

FIG. 3 is a block diagram representation of a procedure for inserting a Jones tube according to the present invention.

### DETAILED DESCRIPTION OF THE INVENTION

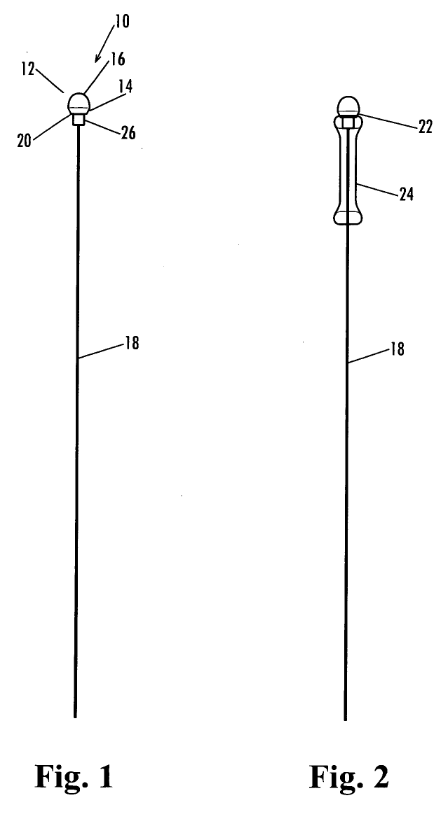

Referring now to the drawing, and in particular to FIG. 1 and FIG. 2, the Jones Tube inserter apparatus of the present invention is approximately 100 mm in length, preferably approximately 115 mm, and is referred to generally by reference numeral 10. It is made of metal, precious metal, plastic, rubber, glass, Teflon or a polymer material.

The distal end or tip 12 is shaped similarly to the Jones Gold Dilator and is from about 0.1 mm to about 5 mm in length. A bullet 14, which is a head 16 similar in shape to a bullet or nosecone with the leading part of the bullet being rounded or oval or other smooth shape, is affixed to one end of a guide line 18 and has similar dimensions as the larger of the two (2) Jones Gold Dilators. The end or base 20 of the bullet is somewhat slanted to accommodate the beveled end 22 of the Jones Tube 24.

At the base of the bullet, opposite the insertion end 16, is guide line 18 (metal, plastic or polymer) that can be affixed to the bullet as a solid apparatus or screwed into the bullet, depending upon the manufacturing process used. Also at the base of the bullet is a small square 26 which is approximately the size of the inside diameter of the Jones Tube. This is to put into the Jones Tube. This keeps the Jones Tube from sliding from one side to the other during insertion.

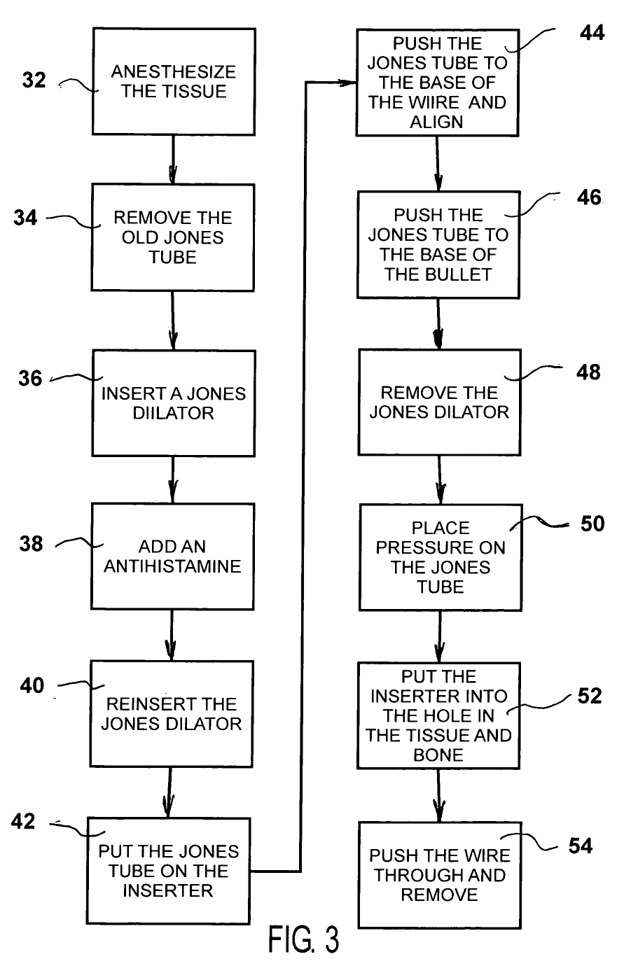

Referring now to FIG. 3, the procedure of the present invention if referred to generally by reference numeral 30 and it includes the following steps:
32 The tissue and surrounding tissue at the site of the conjunctivodacryocystorhinostomy is given anesthetic drops.
34 The old Jones Tube is then removed using forceps. This is relatively easy and causes very little pain or discomfort to the patient.
36 A Jones Gold Dilator, covered with an anesthetic material, is then inserted into the opening of the tissue where the conjunctivodacryocystorhinostomy has been performed and through the hole in the nasal bone to maintain the integrity and to help anesthetize the muscosa of the tissue. The Jones Gold Dilator is removed after about five minutes.
38 An antihistamine is added by means of a Q-tip or other suitable means to shrink the surrounding tissue. It is allowed to set for approximately five (5) minutes.
40 The Jones Gold Dilator is then reinserted into the surgically formed opening, while the Jones Tube is being prepared for insertion.
42 The Jones Tube is put on the inserter.
44 It is pushed to the base of the line. The Jones Tube is then turned to align the beveling in the base of the bullet of the “inserter” with the Jones Tube beveling.
46 The Jones Tube is then pushed towards the bullet so it will be securely held in place by a sleeve that has been placed on the line.
48 The Jones Gold Dilator is then removed.
50 To insert the Jones Tube with the bullet end. The physician places pressure on the Jones Tube by the sleeve that keeps the bullet and Jones Tube seated securely together.
52 The “inserter” is then put into the hole in the tissue and nasal bone. The Jones Tube is then pressed into the tissue free hole in the bone and set into position.
54 Once the Jones Tube has been set, simply push the line through the Jones Tube into the nasal passage and retrieve it with forceps or a magnet or a groove director or other suitable means. Thus the procedure is complete.

The Jones Gold Dilator is inserted into the hole with a fair amount of ease and lack of pain. It is when the Jones Gold Dilator is removed and the line, with the Jones Tube, is inserted, the tissue tends to close in around the line.

If the line has a blunt bullet on the end, similar to that of the Jones Gold Dilator it would push the tissue aside and setting the Jones Tube would not involve the tissue. By doing this procedure using the Jones Tube inserter apparatus of the present invention, it would not be as painful, or as traumatic to the patient.

From the foregoing it will be seen that this invention is well adapted to attain all of the ends and objectives hereinabove set forth, together with other advantages which are inherent to the apparatus.

It will be understood that certain features and subcombinations are of utility and may be employed without reference to other features and subcombinations. This is contemplated by and is within the scope of the claims.

As many possible embodiments may be made of the invention without departing from the scope thereof, it is to be understood that all matter herein set forth or shown in the figures of the accompanying drawings is to be interpreted as illustrative and not in a limiting sense.

### SEQUENCE LISTING

Not Applicable

1. A Jones tube inserter, comprising in combination:
a guide line; and
a bullet affixed to one end of the guide line, wherein the bullet accommodates a Jones tube behind it on the guide line.

2. A Jones tube inserter according to claim 1, wherein the bullet is somewhat slanted to accommodate the beveled end of the Jones tube.

3. A Jones tube inserter according to claim 1, wherein the base of the bullet forms a small square which is approximately the size of the inside diameter of the Jones tube, whereby the square formed by the bullet fits snugly into the Jones tube.

4. A procedure for inserting a Jones tube, comprising in combination the steps of:
a. anesthetize the tissue and surrounding tissue at the site of the conjunctivodacryocystorhinostomy;
b. remove the old Jones Tube, if any;
c. insert a Jones Gold Dilator into the opening of the tissue where the conjunctivodacryocystorhinostomy has been performed and through the hole in the nasal bone to maintain the integrity;
d. add an antihistamine, if necessary, to shrink the surrounding tissue and allowed to set;
e. reinsert the Jones Gold Dilator into the surgically formed opening;
f. put the Jones tube on the inserter;
g. push the Jones tube to the base of the line, and then turn it to align the beveling in the base of the bullet of the inserter with the Jones tube beveling;
h. push the Jones tube towards the bullet so it will be securely held in place by a sleeve during the actual insertion; and
i. remove the Jones Gold Dilator;

5. The procedure of claim 4, further including the step of placing pressure on the Jones tube to keep the bullet and Jones tube seated securely together.

6. The procedure of claim 5, further including the steps of:
putting the “inserter” into the hole in the tissue and nasal bone;
pressing the Jones tube into the tissue free hole in the bone; and
setting the Jones tube into position.

7. The procedure of claim 6, further including the steps of:
pushing the line through the Jones tube into the nasal passage; and
removing the line.
